(19) United States
(12) Patent Application Publication
(10) Pub. No.: US 2009/0192545 A1
(43) Pub. Date: Jul. 30, 2009

(54) Curved suture anchor guide and method of use

(76) Inventor: William Buchanan Workman, Lafayette, CA (US)

Correspondence Address: PATENT LAW OFFICE OF DAVID G. BECK, P. O. BOX 1146, MILL VALLEY, CA, 94942, US

(21) Appl. No.: 12/011,914

(22) Filed: Jan. 30, 2008

(51) Int. Cl. A61B 17/04

(52) U.S. Cl. 606/232

(57) ABSTRACT

A rigid, curved and hollow guide that can be used to facilitate a variety of arthroscopic surgeries is provided as well as a method of using the guide. In one embodiment, the guide (100) is comprised of a shaped hollow guide suitably sized to pass a suture anchor and having a first portion (109) that is straight and a second portion (107) that is curved with a radius of curvature large enough to allow passage of the suture anchor. The guide is further comprised of a handle (103) disposed on the proximal end and a tip (105), preferably angled, on the distal end. In another embodiment, the entire hollow guide (500) is curved.

### FIELD OF THE INVENTION

The present invention relates generally to orthopedic surgery and, more particularly, to a method and apparatus for an improved arthroscopic surgical technique.

### BACKGROUND OF THE INVENTION

Arthroscopy is a diagnostic and surgical technique that has been in use for decades. In its simplest form, it provides a minimally invasive technique for diagnosing any of a variety of disorders, primarily disorders involving a joint such as the knee, shoulder, elbow, hip, ankle and wrist. During diagnosis, one or more small incisions are made around the joint or other area in question and a small, flexible fiberoptic camera, also referred to as an arthroscope, is inserted into the region thereby allowing the physician to inspect and evaluate the injured region while causing minimal damage to surrounding tissues. Once the problem has been assessed, the physician can determine the best course of action, whether that be surgery or a non-surgical approach such as physical and/or drug therapy. If surgery is required, in a large percentage of cases involving the musculoskeletal system the orthopedic surgeon has a choice between using conventional ‘open’ surgical techniques or arthroscopic techniques. The latter approach uses specially designed surgical instruments that can be inserted into small incisions surrounding the region of interest and used to remove and/or repair damaged tissue. As this approach causes minimal damage to surrounding tissue, recovery time is accelerated, post surgery motion is often better than that achievable through open surgery and scarring is reduced.

With the rising popularity of arthroscopy, new techniques and surgical instruments are continually under development with the goal being to achieve higher surgical success rates, lower recurrence rates, and shorter recovery times. As the success and recurrence rates for most joint repair operations are directly tied to the ability of a suture to be properly placed and anchored, suture anchors and suturing systems have been primary areas for active development in recent years.

An exemplary suturing system is disclosed in U.S. Pat. No. 5,342,369, the disclosed system designed to correct anterior shoulder instability by repairing Bankart lesions. The '369 patent discloses a drill and suture guide which is inserted through a posterior portal and between the ball of the humerus and the glenoid cavity. The distal end of the guide is shaped to fit over the glenoid rim in the area of the lesion. Once the guide is properly positioned, a drill bit bores through the glenoid rim. The drill bit is then removed from the guide and replaced with a suture passer. The suture passer is inserted through the recently bored hole in the glenoid rim and then through the detached glenoid labrum. During this process the detached glenoid labrum can be held in place with a tissue grasper inserted through an anterior portal. The suture is then conventionally manipulated and tied, thereby securing the detached glenoid labrum in place.

U.S. Pat. No. 5,683,401 discloses an anchoring system designed to effectively guide suture anchors into place. The '401 patent discloses a hollow grasping instrument which is held in place by grasping tissue at the desired installation site. Once positioned, a threaded suture anchor is passed through the hollow grasper and implanted into the tissue. The hollow grasper is then removed, leaving the suture anchor in place.

U.S. Pat. No. 5,951,559 and related U.S. Pat. No. 5,993,451 disclose a cannulated drill guide and a method of use, the drill guide including an indented tip. The indented or notched tip of the guide is designed to rest on the rim of the glenoid, providing a means of stably holding the guide in place while a suture anchor is installed into the glenoid. As the guide is designed to pass through the ligament, once the threaded suture anchor is seated, the guide can be removed leaving the suture positioned through the ligament.

Although various suture and anchoring systems have been designed, these systems typically require multiple portals in addition to the portal used for the arthroscope. Additionally, in order to properly position the anchor and/or suture in many arthroscopic surgeries, instrument angles are required that lead to larger than desired incisions and cannulas. Accordingly, what is needed in the art is an improved guide and method of use. The present invention provides such a guide and method.

### SUMMARY OF THE INVENTION

The present invention provides a rigid, curved and hollow guide that can be used to facilitate a variety of arthroscopic surgeries. Due to at least a section of the guide being curved, various regions within the surgical site can be easily accessed by simply rotating and/or repositioning the guide. As a result of this capability, fewer and smaller incisions are required during the operation, thus leading to less collateral tissue damage, quicker recovery time and fewer associated cosmetic issues.

In one embodiment of the invention, a multi-use arthroscopic instrument is provided, the instrument comprised of a curved and hollow guide suitably sized to pass a suture anchor and with a sufficiently large radius of curvature to allow passage of the suture anchor and a handle disposed on a proximal end of the hollow guide. The tip of the hollow guide located at the distal end of the guide can be angled. In one configuration, the radius of curvature is greater than or equal to 12.5 centimeters. In an alternate configuration, the radius of curvature is greater than or equal to 5.5 centimeters. In yet another configuration, the radius of curvature is greater than or equal to 3.0 centimeters.

In another embodiment of the invention, a multi-use arthroscopic instrument is provided, the instrument comprised of a shaped hollow guide suitably sized to pass a suture anchor and a handle disposed on a proximal end of the hollow guide. The tip of the hollow guide located at the distal end of the guide can be angled. The guide shaft of the shaped hollow guide has a first portion proximate to the handle and a second portion proximate to the guide tip, wherein the first portion is straight and the second portion is curved with a sufficiently large radius of curvature to allow passage of the suture anchor. In one configuration, the radius of curvature is greater than or equal to 12.5 centimeters. In an alternate configuration, the radius of curvature is greater than or equal to 5.5 centimeters. In yet another configuration, the radius of curvature is greater than or equal to 3.0 centimeters.

In yet another embodiment of the invention, a method for installing a suture anchor for securing tissue to a bone is provided, the method comprised of the steps of locating a tip of a rigid, curved and hollow guide in proximity to the arthroscopic surgical site, the site including the desired location for the suture anchor; inserting a preparatory tool through the rigid, curved and hollow guide; preparing the surgical site with the preparatory tool; penetrating the soft tissue to be secured to the bone with the rigid, curved and hollow guide; positioning the tip of the rigid, curved and hollow guide at the desired suture anchor location; inserting a suture anchor and the anchor driver into the rigid, curved and hollow guide; positioning the suture anchor at the desired anchor location; driving the suture anchor into the bone with the anchor driver; withdrawing the rigid, curved and hollow guide from the soft tissue; and securing the soft tissue to the suture anchor and the bone with a suture coupled to the suture anchor. A tissue-penetrating tool can be inserted through the rigid, curved and hollow guide prior to penetrating the soft tissue with the rigid, curved and hollow guide. The method can further be comprised of the step of rotating the rigid, curved and hollow guide after penetrating the soft tissue, but before positioning the tip at the desired suture anchor location. The soft tissue may be comprised of articular capsule and glenoid labrum tissue. The rigid, curved and hollow guide may be inserted through a portal, the portal not extending through the soft tissue. The method can additionally be comprised of the step of inserting a flexible drill through the curved and hollow guide and drilling an anchor hole at the desired anchor location. The method can additionally be comprised of the step of pre-threading the suture anchor.

A further understanding of the nature and advantages of the present invention may be realized by reference to the remaining portions of the specification and the drawings.

### BRIEF DESCRIPTION OF THE DRAWINGS

FIG. 1 is a side view of a hollow and curved guide in accordance with a preferred embodiment of the invention;

FIG. 2 is a cut-away view of the hollow and curved guide shown in FIG. 1;

FIG. 3 is a side view of a hollow and curved guide similar to that shown in FIG. 1 except for the use of a T-shaped handle;

FIG. 4 is a side view of a hollow and curved guide similar to that shown in FIG. 1 except that it includes means for identifying the direction of curvature of the guide shaft;

FIG. 5 is a side view of a hollow and curved guide in accordance with an alternate embodiment of the invention;

FIG. 6 is a cut-away view of an alternate configuration in which the curved guide passes through a non-curved handle;

FIG. 7 is a simplified illustration of a Bankart lesion with the hollow and curved guide of the invention in place;

FIG. 8 is an illustration of a flexible tool for use with the hollow and curved guide of the invention;

FIG. 9 is an illustration of a curved tool for use with a curved guide such as those shown in FIGS. 5 and 6;

FIG. 10 is an illustration of a tool tip that can be integrated into tools for use with the curved guide of the invention; and

FIG. 11 is an illustration of an alternate tool tip that can be integrated into tools for use with the curved guide of the invention.

### DESCRIPTION OF THE SPECIFIC EMBODIMENTS

The method and apparatus of the present invention can be used to facilitate a variety of arthroscopic surgeries, and has been found to be of particular use in repairing Bankart lesions. Accordingly, although some of the procedural descriptions provided herein are specific to Bankart lesions, it will be understood that the applicability of the present invention is not limited to arthroscopic Bankart repairs.

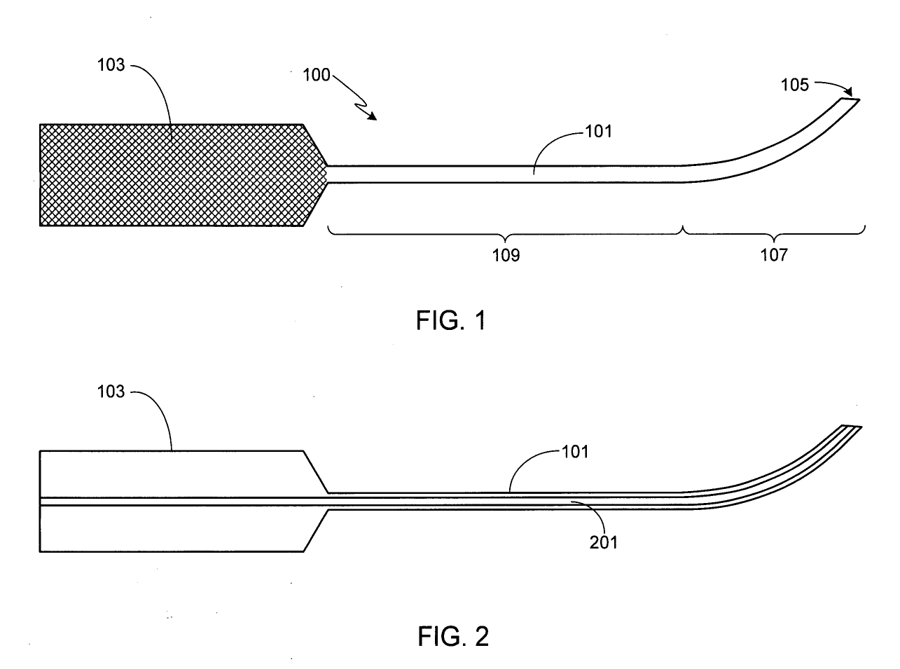

FIG. 1 is a side view of a rigid, curved and hollow guide 100 in accordance with a preferred embodiment of the invention. Guide 100 is comprised of a hollow guide shaft 101 coupled to a handle 103. As shown in the cut-away view of FIG. 2, the hollow guide portion 201 extends through both guide shaft 101 and handle 103, thus allowing sutures, suture anchors, and various tools to be passed through guide 100, for example as described in further detail below. Preferably tip 105 of shaft 101 is angled, more preferably with a tip angle of approximately 45 degrees.

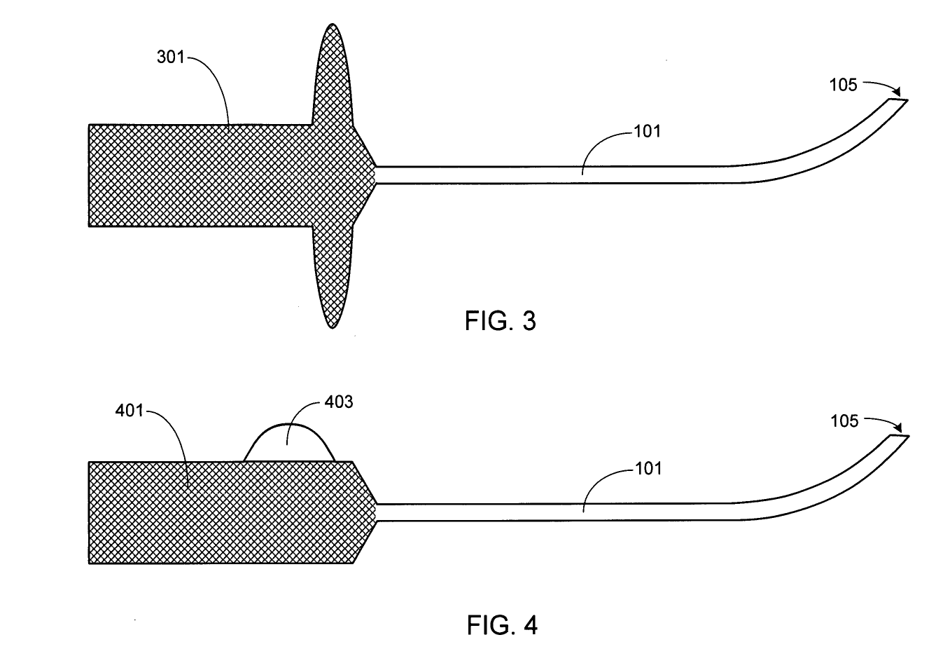

In the illustrated embodiment, handle 103 is knurled, thus providing a non-slip surface that can be easily grasped and turned as necessary during surgery. It will be appreciated that other handle shapes and styles can also be used with the invention. For example, handle 301 in FIG. 3 is a T-shaped handle, thus providing the surgeon with an easily grasped handle that aids during the manipulation and rotation of the curved guide. In at least one embodiment of the invention, the handle includes means of identifying the direction of curvature of guide shaft 101. Exemplary identification means include applying identification markings to the handle and shaping the handle. For example, in an embodiment of the invention illustrated in FIG. 4, guide handle 401 includes a tab 403 that extends away from the handle and is aligned with the curvature of shaft 101, tab 403 providing a means of identifying the direction of guide curvature as well as simplifying guide manipulation.

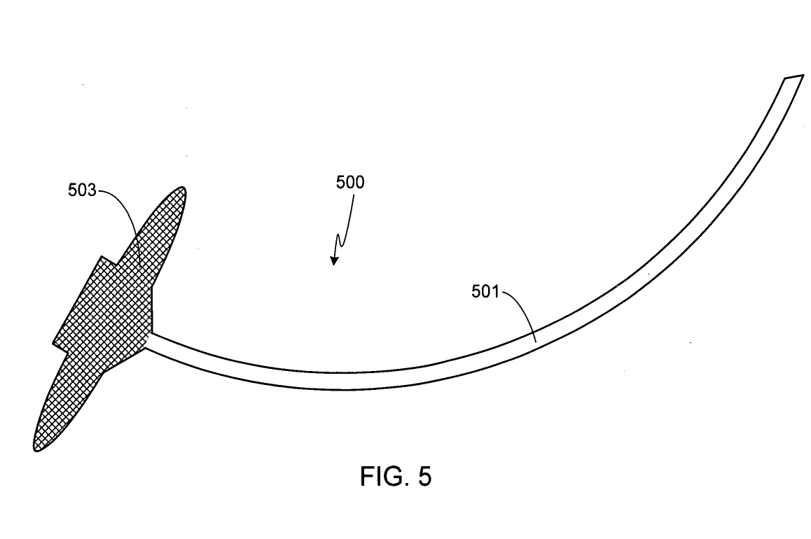

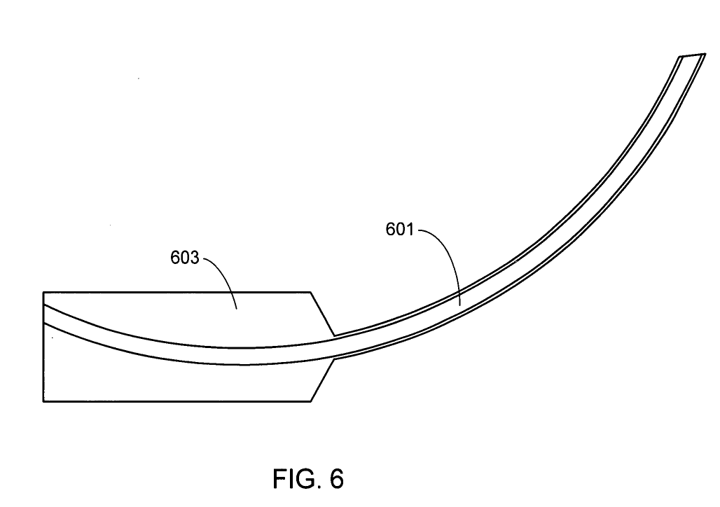

In the embodiments illustrated in FIGS. 1-4, only an end portion 107 of guide shaft 101 is curved, the remaining portion 109 being straight. Utilizing this configuration, guides can be manufactured with different lengths for portion 107. In the preferred embodiment the length of portion 107 is between 10 centimeter and 20 centimeters, and more preferably, approximately 15 centimeters. Due to the inclusion of both straight and curved portions, i.e., portions 109 and 107 respectively, the configurations illustrated in FIGS. 1-4 require that all tools passing through the guide be flexible. To overcome this limitation, in at least one embodiment of the invention the entire guide, including the portion passing through the handle, is curved. An example of such a rigid, curved and hollow guide is shown in FIG. 5. As shown, the entire guide shaft 501 of guide 500 is curved. Preferably in this configuration handle 503 is kept relatively short since manipulation of a curved handle is somewhat awkward. Alternately, and as illustrated in the cut-away view of FIG. 6, hollow guide portion 601 can remain curved as it passes through a non-curved handle 603.

Regardless of whether the entire guide or only a portion of the guide is curved, the radius of curvature cannot be too small since the guide must be able to pass suture anchors and, in applications in which the anchor requires pre-drilling of the anchor site, a flexible drill. Under the assumption that a flexible portal is used, the sharpest allowable radius of curvature for the curved guide (e.g., portion 107 or entire guide 501) depends on the outer diameter (OD) and the length of the longest rigid structure that the surgeon wishes to pass through the guide. Typically this structure is either the end portion of a drill bit, assuming the surgeon intends to drill a hole prior to anchor placement, or the anchor itself. If a rigid portal is used, additional factors that must be considered in determining the allowable radius of curvature for the guide are the inner diameter (ID) of the portal, the length of the portal, and the OD of the guide shaft. Note that in use, some surgeons may prefer to have multiple guides available, each with a different radius of curvature, thus providing multiple options after the surgeon has had an opportunity to arthroscopically evaluate the surgical site.

As previously noted, the smallest allowable radius of curvature can be determined from the largest rigid structure that needs to be passed through the guide. For example, assuming a curved guide with an ID of 2.5 millimeters, and the maximum rigid structure being an anchor with an OD of 2.3 millimeters and a length of 12 millimeters, and assuming a manufacturing tolerance of ±1 percent, the smallest allowable radius of curvature is approximately 12.5 centimeters. Alternately, keeping all values the same except for anchor length, and using an anchor with a length of 8 millimeters, the smallest allowable radius of curvature is approximately 5.5 centimeters. Shortening the anchor even further to a length of 6 millimeters allows the radius of curvature to be decreased even further to approximately 3.0 centimeters. It will be appreciated that these values for the radius of curvature for the guide are only meant to be illustrative and that the actual value can be easily calculated once the maximum dimensions of the rigid structure, the desired ID of the guide, and the manufacturing tolerances are known. Additionally and as previously noted, if a rigid portal is used, or if the portal has limited flexibility, then the radius of curvature also depends on the OD of the guide, the ID/length of the portal, and the flexibility of the portal.

The curved guide of the present invention offers a number of advantages over prior art guides. First, it provides access to regions that would not be accessible, or at angles that would not be achievable, with a non-curved guide. For example, when performing an arthroscopic Bankart repair, it is critical that the labrum and capsular tissue be properly positioned at the 5-6 o'clock position on the glenoid (right shoulder). Unfortunately, a straight pass through the capsular tissue is typically prevented due to the location of the axillary nerve. Accordingly, a curved guide and tissue penetrator as presently disclosed provides superior access to this region. A second advantage of the curved guide is that it provides access to more regions within the surgical site than are achievable using a non-curved guide, the additional access being provided through rotating and repositioning the guide. As a result of this capability, fewer and smaller incisions are required during an operation, thus leading to less collateral tissue damage, quicker recovery, and fewer cosmetic issues.

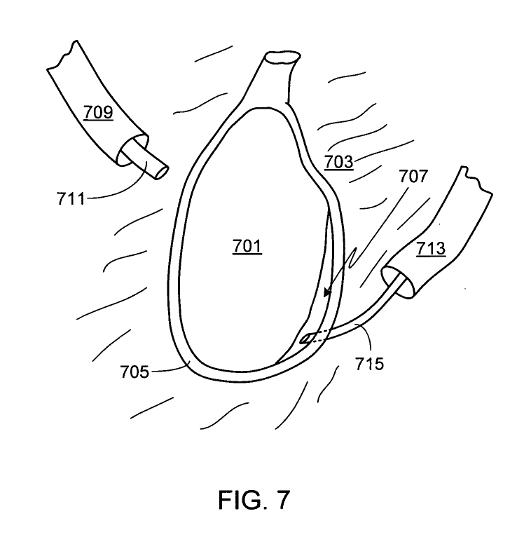

An exemplary method of using the curved guide of the invention will now be described in detail. FIG. 7 is a simplified illustration of a Bankart lesion. More specifically, FIG. 7 shows the glenoid cavity 701 encircled by the articular capsule 703 which is attached at the margin of the cavity by the glenoid labrum 705. As shown in this figure, the glenoid labrum 705 has been partially torn from the rim of the glenoid cavity 701, creating Bankart lesion 707.

Initially, and following standard protocols, a first portal 709 is opened allowing passage of an arthroscope 711 and permitting initial evaluation. Although a posterior portal 709 is shown, it will be understood that in some instances an anterior portal will be used for the arthroscope. After evaluation, an anterior portal 713 is opened through which a curved guide 715 is inserted. Although the use of a portal in conjunction with the curved guide is preferred, it will be appreciated that curved guide 715 can be inserted directly through the incision, typically combining guide 715 with an obturator, thereby eliminating the need for portal 713.

Prior to penetration of the soft tissue (e.g., articular capsule and glenoid labrum), the surgical site must be properly prepared. Typically this preparation step requires first liberating any tissue remnants attached to the glenoid rim in the area of the lesion and then prepping the surface of the glenoid rim. The first part of this step can be performed with any of a variety of tools designed to pass through the curved guide while the second part of the step is preferably performed with a rasp, also designed to pass through the curved guide. It will be appreciated that if the curved guide includes both straight and curved portions as shown in FIGS. 1-4, the tools for this step will need to be flexible. Of course the tools can include one or more non-flexible regions, for example at the tool end, as long as the non-flexible region(s) is no longer than that allowed by the radius of curvature of the guide as previously described. Accordingly, the non-flexible regions of these tools will typically be no longer than the intended suture anchors. If the curved guide has a continuous curve as in the embodiments shown in FIGS. 5 and 6, either the tools must be flexible as described above, or rigid with a radius of curvature that matches that of the curved guide.

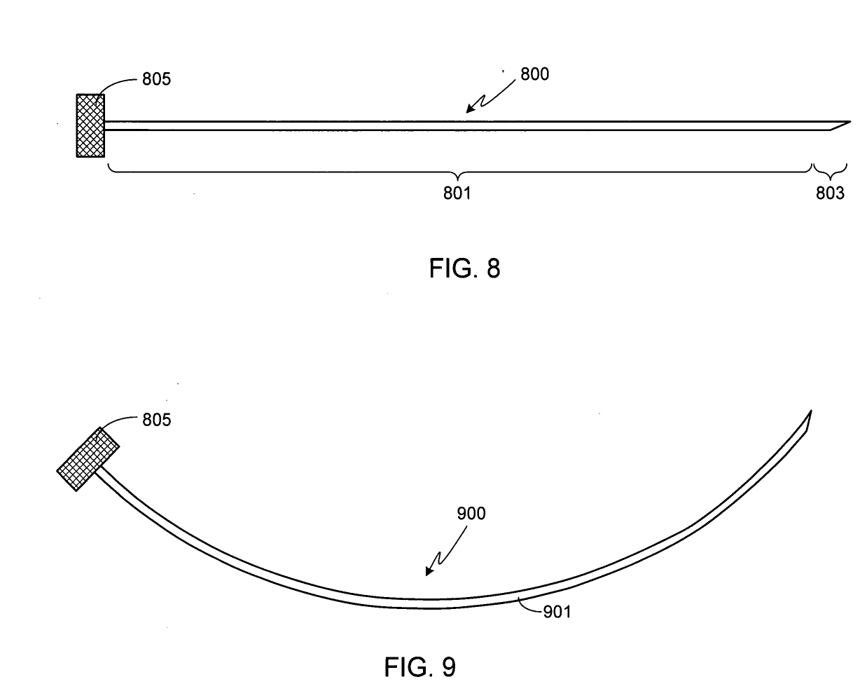

FIGS. 8-11 illustrate a few suitable tools for use during the site preparation step described above. It will be appreciated that these are simply examples of suitable tools and that the invention is not limited to the use of these tools during the site preparation step. For example, most arthroscopic tools in standard use today can be redesigned to include a flexible shaft or a curved shaft of suitable radius of curvature, thus allowing these tools to be used with the curved guide of the invention.

FIG. 8 is an example of a tool that can fit into any of the embodiments shown in FIGS. 1-6. Although the entire shaft of tool 800 can be designed to be flexible, preferably tool 800 includes a flexible shaft portion 801 and a rigid tip portion 803, thus providing improved tool tip control during the site preparation step. As previously noted, the rigid portion 803 of the tool must be short enough to slide through the curved guide. Accordingly, either the radius of curvature of the curved guide must be selected based on the length of rigid portion 803, or the length of rigid portion 803 must be no longer than that for which the curved guide was designed.

FIG. 9 is an example of a tool that can be used instead of tool 800 for the embodiments of the curved guide shown in FIGS. 5 and 6. Shaft 901 of tool 900 has the same radius of curvature as the curved guide with which it is to be used.

Tools designed for use with the curved guide are long enough to insure that their shafts extend past the handle of the curved guide during use. A tool handle 805 is preferably provided on at least a portion of the tool shaft that extends past the handle of the curved guide, handle 805 providing tool control. In the exemplary tools, handle 805 is a knurled handle.

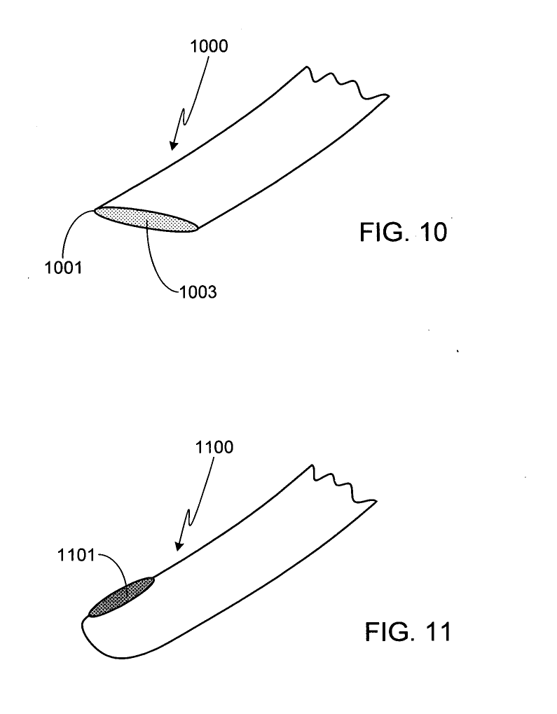

FIGS. 10 and 11 provide illustrations of two exemplary tool tips that can be integrated into the tools shown in FIGS. 8 and 9. Tool tip 1000 of FIG. 10 is angled, thus providing chisel-like functionality. Edge 1001 of tip 1000 is sharpened to aid in tissue liberation while the central region 1003 is configured with an abrading surface. In alternate exemplary tool tip 1100 shown in FIG. 11, the tip includes a shaver surface 1101.

The next step is to penetrate the soft tissue with curved guide 715 at the location where the first suture is desired. For a Bankart repair, typically the first penetration of capsule 703 and labrum 705 is near the 5 o'clock position since it is critical that this region is properly secured in order to gain the desired level of stability and mobility. During this step, the curve of the curved guide 715 provides a distinct advantage over a non-curved guide since the curvature provides relatively easy access to the desired penetration point, going around the axillary nerve.

Although the tip of curved guide 715 can be angled and sharpened sufficiently to allow it to directly penetrate the soft tissue (e.g., the articular capsule and glenoid labrum), this is not preferred for several reasons. First, if the tip angle is too acute, the tip will not rest easily on the bone during the anchor location and placement steps. Second, if the edges of the guide tip are too sharp, there is a greater risk of the guide cutting or otherwise damaging the bone during guide manipulation and anchor location/placement. Accordingly, in the preferred embodiment of the invention, a tool with a suitable penetrating tip, such as tip 1000, is inserted into curved guide 715 during the tissue-penetrating step. This tool also performs the function of an obturator, i.e., preventing tissue from becoming lodged within the curved guide during the tissue-penetrating step.

After the soft tissue has been penetrated, the penetration tool is removed from the curved guide, assuming such a tool was used as is preferred, and the tip of the curved guide is positioned for placement of the first suture anchor. The inventor has found that by rotating the curved guide, preferably by approximately 180 degrees, a proper anchor insertion angle is achieved.

It will be appreciated that the exact steps required to install a suture anchor depends upon the type of suture anchor being used. For example in a preferred embodiment, after properly positioning the curved guide at the anchor site, a flexible drill is inserted through the curved guide and used to drill an anchor hole. The drill is then removed from the curved guide and replaced with the suture anchor and an anchor driver, the anchor driver configured to fit through the curved guide as described above relative to tools 800 and 900. Preferably the suture anchor is pre-threaded with either single or multiple sutures.

In an alternate embodiment, a suture anchor that does not require pre-drilling is used. In this embodiment the suture anchor, preferably pre-threaded with single or multiple sutures, is placed within the guide along with a suitable driver (e.g., a screw driver) that is configured to fit through the curved guide. If the driver is required to rotate during anchor setting, for example in order to screw the anchor into place, then the driver is flexible, thus allowing it to rotate within the curved guide.

The final steps of the process require a suitable knot to be tied in the suture and then the excess suture material to be cut. As the method of the invention properly locates the anchor and passes the suture through the desired location in the soft tissue (e.g., articular capsule and glenoid labrum) using a single curved guide, the next step requires that the curved guide be withdrawn, at least withdrawn from the soft tissue, and any of a variety of suture retrievers be used to retrieve one leg of the suture. Using the leg of the suture that had been passed through the tissue using the curved guide and the second leg retrieved with the suture retriever, a knot is tied in the suture, thereby tying the tissue down to the anchor and thus the glenoid. As any of a variety of conventional tools well known by those of skill in the art can be used to retrieve the second leg of the suture, tie the suture knot, and cut the excess suture material, further details of these instruments will not be provided herein. After securing the tissue to the anchor, and thus the bone in which the anchor is attached, the process is repeated for the next anchor (i.e., penetrating the tissue at the desired site, positioning the guide, installing the next anchor, and tying/cutting the suture).

Accordingly, the process described above allows the suture anchor to be set and the suture to be passed through the soft tissue (e.g., articular capsule and glenoid labrum) without requiring the use of a separate suture passer, thereby eliminating this step. Additionally, due to the use of the curved guide, incision size and number are minimized while still providing means for optimally locating the anchor and suture.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention that is set forth in the following claims.

1. A multi-use arthroscopic instrument comprising:
a continuously curved hollow guide suitably sized to pass a suture anchor, wherein a radius of curvature defining said continuously curved hollow guide is sufficiently large to allow passage of said suture anchor; and
a handle disposed on a proximal end of said continuously curved hollow guide, wherein said continuously curved hollow guide passes through said handle.

2. The multi-use arthroscopic instrument of claim 1, further comprising an angled tip at a distal end of said continuously curved hollow guide.

3. The multi-use arthroscopic instrument of claim 1, wherein said radius of curvature is sufficiently large to allow passage of said continuously curved hollow guide through a portal.

4. The multi-use arthroscopic instrument of claim 1, wherein said radius of curvature is greater than or equal to 12.5 centimeters.

5. The multi-use arthroscopic instrument of claim 1, wherein said radius of curvature is greater than or equal to 5.5 centimeters.

6. The multi-use arthroscopic instrument of claim 1, wherein said radius of curvature is greater than or equal to 3.0 centimeters.

7. The multi-use arthroscopic instrument of claim 1, wherein said handle further comprises means for identifying a direction of curvature of said continuously curved hollow guide.

8. A multi-use arthroscopic instrument comprising:
a shaped hollow guide suitably sized to pass a suture anchor;
a handle disposed on a proximal end of said hollow guide, wherein said hollow guide passes through said handle;
an angled tip at a distal end of said shaped hollow guide; and
wherein a guide shaft of said shaped hollow guide has a first portion proximate to said handle and a second portion proximate to said angled tip, wherein said first portion is straight and said second portion is curved, and wherein a radius of curvature defines said curved second portion and is sufficiently large to allow passage of said suture anchor.

9. The multi-use arthroscopic instrument of claim 8, wherein said angled tip has an angle of approximately 45 degrees.

10. (canceled)

11. (canceled)

12. (canceled)

13. (canceled)

14. The multi-use arthroscopic instrument of claim 8, wherein said handle further comprises means for identifying a direction of curvature of said second portion of said guide shaft.

15. A method for installing a suture anchor for securing soft tissue to bone, the method comprising the steps of:
locating a tip of a rigid, curved and hollow guide in proximity to an arthroscopic surgical site, said arthroscopic surgical site including a desired suture anchor location;
inserting a preparatory tool through said rigid, curved and hollow guide;
preparing said surgical site with said preparatory tool;
penetrating the soft tissue to be secured to the bone with said rigid, curved and hollow guide;
positioning said tip of said rigid, curved and hollow guide at said desired suture anchor location;
inserting the suture anchor into said rigid, curved and hollow guide;
inserting an anchor driver into said rigid, curved and hollow guide;
positioning said suture anchor at said desired suture anchor location;
driving said suture anchor into the bone with said anchor driver;
withdrawing said rigid, curved and hollow guide from said soft tissue; and
securing the soft tissue to the suture anchor and the bone with a suture coupled to the suture anchor.

16. The method of claim 15, further comprising the step of withdrawing said preparatory tool from said rigid, curved and hollow guide prior to said soft tissue penetrating step.

17. The method of claim 15, further comprising the step of inserting a penetrating tool through said rigid, curved and hollow guide, wherein said step of inserting said penetrating tool is performed prior to said soft tissue penetrating step.

18. The method of claim 15, further comprising the step of rotating said rigid, curved and hollow guide prior to said tip positioning step.

19. The method of claim 18, wherein said rotating step further comprises the step of rotating said rigid, curved and hollow guide by approximately 180 degrees.

20. The method of claim 15, wherein said soft tissue includes articular capsule and glenoid labrum tissue.

21. The method of claim 15, further comprising the step of inserting said rigid, curved and hollow guide through a portal, wherein said portal does not extend through said soft tissue, and wherein said step of inserting said rigid, curved and hollow guide through said portal is performed prior to said tip locating step.

22. The method of claim 15, further comprising the steps of inserting a flexible drill through said rigid, curved and hollow guide, and drilling an anchor hole at said desired anchor location with said flexible drill, wherein said flexible drill inserting step and said anchor hole drilling step are performed prior to said suture anchor inserting step.

23. The method of claim 15, wherein said suture anchor driving step further comprises the steps of rotating said anchor driver and screwing said suture anchor into the bone at said desired anchor location.

24. The method of claim 15, further comprising the step of pre-threading said suture anchor with said suture, wherein said pre-threading step is performed prior to said suture anchor inserting step.
